(19) United States
(12) Patent Application Publication
(10) Pub. No.: US 2021/0163764 A1
(43) Pub. Date: Jun. 3, 2021

(54) STABILIZATION OF DCOIT IN AQUEOUS SYSTEMS

(71) Applicant: DDP Specialty Electronic Materials US, Inc., Collegeville, PA (US)

(72) Inventors: Timothy De Vries, Collegeville, PA (US); Stacey Barnaby, Collegeville, PA (US); Andrew Camelio, Collegeville, PA (US); Kevin Lawry, Collegeville, PA (US); Daniel Miller, Collegeville, PA (US); Hiteshkumar Dave, Collegeville, PA (US); Marcos Garcia-Bernardi, Collegeville, PA (US)

(21) Appl. No.: 16/769,012

(22) PCT Filed: Dec. 21, 2018
(86) PCT No.: PCT/US2018/067094

§ 371 Date: Jun. 2, 2020

Related U.S. Application Data
(60) Provisional application No. 62/611,113, filed on Dec. 28, 2017.

(51) Int. Cl. C09D 7/63; C07D 275/03; C09D 133/04; C09D 7/48

(52) U.S. Cl. CPC C09D 7/63; C07D 275/03; C08K 5/47; C09D 7/48; C09D 133/04

(57) ABSTRACT

An aqueous composition comprising: (a) 4,5-dichloro-2-n-octyl-4-isothiazolin-3-one; and (b) an aromatic acid anhydride comprising from 6 to 30 carbon atoms.

### BACKGROUND

This invention generally relates to stabilized aqueous compositions comprising 4,5-dichloro-2-n-octyl-4-isothiazolin-3-one (DCOIT).

The use of DCOIT in aqueous compositions, including e.g., paints is known. U.S. Pat. No. 5,198,455 discloses isothiazolone formulations comprising organic acid anhydrides. However, methods of stabilizing DCOIT in aqueous compositions are not always effective, can cause discoloration, and/or are not compatible with coatings compositions.

The problem solved by the present invention is to provide additional methods for stabilizing aqueous compositions comprising DCOIT.

### STATEMENT OF THE INVENTION

The present invention is directed to an aqueous composition comprising: (a) 4,5-dichloro-2-n-octyl-4-isothiazolin-3-one; and (b) an aromatic acid anhydride comprising from 6 to 30 carbon atoms.

The present invention is further directed to an aqueous composition comprising: (a) 4,5-dichloro-2-n-octyl-4-isothiazolin-3-one; and (b) a solid aliphatic acid anhydride comprising from 4 to 32 carbon atoms.

### DETAILED DESCRIPTION OF THE INVENTION

All percentages are weight percentages (wt %), all fractions are by weight and all temperatures are in ° C., unless otherwise indicated. Unless otherwise specified all operations are performed at room temperature (20-25° C.). Compounds referred to as “solid” have a melting point at normal atmospheric pressure (101 kPa) of at least 40° C. (preferably at least 70° C.). Percentages of polymerized monomer units in polymers are based on the entire weight of the solid polymer, i.e., excluding water or solvents. Any term containing parentheses refers, alternatively, to the whole term as if no parentheses were present and the term without them, and combinations of each alternative. Thus, the term “(meth)acrylic” refers to any of acrylic, methacrylic, and mixtures thereof. A “C₃-C₆ carboxylic acid monomer” is a mono-ethylenically unsaturated compound having one or two carboxylic acid groups, e.g., (meth)acrylic acid, maleic acid, fumaric acid, itaconic acid, maleic anhydride, crotonic acid, etc. Preferably, the acid monomer has three or four carbon atoms, preferably one carboxylic acid group, preferably (meth)acrylic acid, preferably methacrylic acid (MAA). Alkyl groups are saturated hydrocarbyl groups which may be straight or branched. The term “aromatic” includes heterocyclic aromatic compounds, e.g., pyridines, pyrroles, as well as hydrocarbyl aromatic compounds, e.g., benzenes and naphthalenes.

Crosslinkers are monomers having two or more non-conjugated ethylenically unsaturated groups. Preferred crosslinkers include, e.g., di- or tri-allyl ethers and di- or tri-(meth)acrylyl esters of diols or polyols (e.g., trimethylolpropane diallyl ether (TMPDAE) and trimethylolpropane trimethacrylate (TMPTMA)), di- or tri-allyl esters of di- or tri-acids, allyl (meth)acrylate, divinyl sulfone, triallyl phosphate, divinylaromatics (e.g., divinylbenzene). Preferably, the polymer comprises no more than 0.5 wt % polymerized units of crosslinkers, preferably no more than 0.2 wt %, preferably no more than 0.1 wt %, preferably no more than 0.05 wt %.

Preferably, the aqueous composition comprises a dispersed polymer, preferably an emulsion polymer. Preferably, the polymer is an acrylic, acrylic-styrenic, or polyvinyl acetate polymer. A polyvinyl acetate polymer comprises at least 30 wt % polymerized units of polyvinyl acetate, preferably at least 40 wt %, preferably at least 50 wt %, preferably at least 60 wt %. Polyvinyl acetate polymers may contain polymerized units of acrylic and/or styrenic monomers. An acrylic-styrenic polymer comprises at least 70 wt % polymerized units selected from acrylic monomers and styrenic monomers, preferably at least 80 wt %, preferably at least 90 wt %, preferably at least 95 wt %, preferably at least 98 wt %, preferably at least 99 wt %. An acrylic polymer comprises at least 70 wt % polymerized units selected from acrylic monomers, preferably at least 80 wt %, preferably at least 90 wt %, preferably at least 95 wt %, preferably at least 98 wt %, preferably at least 99 wt %. Acrylic monomers include (meth)acrylic acids and their esters; crotonic acid, itaconic acid, fumaric acid, maleic acid, maleic anhydride, (meth)acrylamides, (meth)acrylonitrile and esters of crotonic acid, itaconic acid, fumaric acid or maleic acid. The acrylic polymer may also comprise other polymerized monomer units including, e.g., vinyl esters of C₁-C₂₂ alkyl carboxylic acids, vinyl amides (including, e.g., N-vinylpyrrolidone), sulfonated acrylic monomers, vinyl sulfonic acid, vinyl halides, phosphorus-containing monomers, heterocyclic monomers and styrenic monomers. Styrenic monomers include styrene, 4-methylstyrene, 2-methylstyrene, 3-methylstyrene, 4-methoxystyrene, 2-hydroxymethylstyrene, 4-ethylstyrene, 4-ethoxystyrene, 3,4-dimethyl styrene, 2-chlorostyrene, 3-chlorostyrene, 4-chloro-3-methyl styrene, 4-t-butyl styrene, 2,4-dichlorostyrene, 2,6-dichlorostyrene and vinylnaphthalene; preferably styrene. In a preferred embodiment of the invention the polymer is an acrylic polymer.

Preferably, the polymer comprises at least 70 wt % polymerized units of monomers selected from (meth)acrylic acid, C₁-C₁₂ alkyl (meth)acrylates and styrenic monomers; preferably, at least 75 wt %, preferably at least 80 wt %, preferably at least 85 wt %, preferably at least 90 wt %, preferably at least 95 wt %. Preferably, C₁-C₁₂ alkyl (meth)acrylates are limited to a C₁-C₈ alkyl (meth)acrylates, preferably C₁-C₆ alkyl (meth)acrylates, preferably C₁-C₄ alkyl (meth)acrylates. Preferably the polymer comprises from 5 to 20 wt % polymerized units of (meth)acrylic acid, preferably at least 7 wt %, preferably at least 8 wt %; preferably no more than 17 wt %, preferably no more than 14 wt %. Preferably, the polymer comprises from 1 to 20 wt % polymerized units of styrenic monomers, preferably at least 3 wt %, preferably at least 5 wt %, preferably at least 7 wt %; preferably no more than 17 wt %, preferably no more than 15 wt %. In a preferred embodiment, the polymer comprises at least 70 wt % polymerized units of monomers selected from (meth)acrylic acid and C₁-C₁₂ alkyl (meth)acrylates; preferably, at least 75 wt %, preferably at least 80 wt %, preferably at least 85 wt %, preferably at least 90 wt %, preferably at least 95 wt %.

Preferably, the aqueous composition comprises the polymer as discrete particles dispersed in an aqueous medium, i.e., a polymer latex. In this aqueous dispersion, the average particle diameter of the polymer particles preferably is in the range from 50 to 2000 nm, preferably from 100 to 1000 nm, preferably from 150 to 800 nm. The level of polymer particles in the aqueous dispersion is typically in the range of from 15 to 60 wt %, preferably 25 to 50 wt %, based on the weight of the aqueous dispersion. Preferably, the concentration of DCOIT in the aqueous composition is from 0.01 to 2 wt %; preferably at least 0.05 wt %, preferably at least 0.08 wt %; preferably no more than 1 wt %, preferably no more than 0.5 wt %, preferably no more than 0.15.

Preferably, the aromatic acid anhydride is an anhydride of a carboxylic or polycarboxylic acid. Preferably, the aromatic acid anhydride comprises at least 7 carbon atoms, preferably at least 8; preferably no more than 20 carbon atoms, preferably no more than 15, preferably no more than 12. Particularly preferred aromatic acid anhydrides include phthalic anhydride, pyromellitic dianhydride and benzoic anhydride. Preferably, the mole ratio of the anhydride to DCOIT is from 1:4 to 1:24; preferably at least 1:6, preferably at least 1:8; preferably no more than 1:20, preferably no more than 1:18, preferably no more than 1:12. Preferably the anhydride is added to the aqueous composition prior to or together with DCOIT.

Preferably, the aliphatic acid anhydride is an anhydride of an aliphatic carboxylic or dicarboxylic acid. Preferably, the aliphatic acid anhydride has no more than 25 carbon atoms, preferably no more than 22, preferably no more than 19, preferably no more than 12, preferably no more than 8. In a preferred embodiment, the aliphatic acid anhydride is an anhydride of a maleic or succinic acid. Especially preferred anhydrides of maleic and succinic acids include maleic anhydride, succinic anhydride and 2,3-dichloromaleic anhydride. In a preferred embodiment, the anhydride is an alkyl- or alkenyl-substituted succinic acid anhydride, preferably one having only a single alkyl or alkenyl group. Preferably, the alkyl or alkenyl group has at least 8 carbon atoms, preferably at least 12 carbon atoms, preferably at least 14 carbon atoms. Preferably, the mole ratio of the anhydride to DCOIT is from 1:2 to 1:34; preferably at least 1:5, preferably at least 1:8, preferably at least 1:10; preferably no more than 1:30, preferably no more than 1:20, preferably no more than 1:18. Preferably, the aliphatic acid anhydride is added to an aqueous composition comprising DCOIT.

The present invention is further directed to an aqueous composition comprising: (a) 4,5-dichloro-2-n-octyl-4-isothiazolin-3-one; and (b) an aromatic imide comprising from 6 to 30 carbon atoms. Preferably, the aromatic imide is an imide of an aromatic dicarboxylic or polycarboxylic acid. Preferably, the aromatic imide comprises at least 7 carbon atoms, preferably at least 8; preferably no more than 20 carbon atoms, preferably no more than 15, preferably no more than 12. Particularly preferred aromatic imides include phthalimide, pyromellitic diimide, 4-methylphthalimide; 2,3-Naphthalenedicarboximide; 4- or 5-Methoxyisoindoline-1,3-dione; 3- or 4-Aminophthalimide; 4-bromo-, 4-chloro-, or 4-fluorophthalimide; 5-(tert-Butyl)isoindoline-1,3-dione. Preferably, the mole ratio of the anhydride to DCOIT is from 1:2 to 1:34; preferably at least 1:5, preferably at least 1:8, preferably at least 1:10; preferably no more than 1:30, preferably no more than 1:20, preferably no more than 1:18. Preferably, the aromatic imide is added to an aqueous composition comprising DCOIT.

Typical aqueous emulsion polymerization techniques are suitable for preparation of the acrylic polymer. Aqueous emulsion polymerization processes typically are conducted in an aqueous reaction mixture, which contains at least one monomer and various synthesis adjuvants such as the free radical sources, buffers, and reductants in an aqueous reaction medium. Preferably, polymerization is carried out to obtain random copolymers. Preferably, the aqueous emulsion polymer has Mw from 100,000 to 2,000,000; preferably at least 300,000, preferably at least 400,000; preferably no more than 1,500,000.

The emulsion of aqueous acrylic polymer particles are useful in coatings applications, e.g., (architectural coatings, etc.), roof coatings, wood finishes (e.g., wood stains and other protective finishes for wood), industrial coatings, and specialty coatings (automotive, industrial maintenance, traffic marking, and marine). In a preferred embodiment of the invention, the aqueous emulsion of acrylic, styrene-acrylic or polyvinyl acetate polymer is a latex paint or semi-transparent stain which contains inorganic hiding and extender pigment(s) such as titanium dioxide, kaolin, talc, calcium carbonate, nepheline syenite, barite, mica or silica. Preferred pigment ranges are 2-50 wt %, preferably 10-45%.

### Examples

### Inhibitor Testing:

A stock solution of binder RHOPLEX™ HG-706 (100 g; The Dow Chemical Company; 100% acrylic latex binder)+BIOBAN™ 200 (0.50 g; The Dow Chemical Company)+anisole (50 μL, as an internal standard) was prepared. A 10 g portion was held at room temperature for 7-9 days; a second 10 g portion was heated to 50° C. in a water bath for the same amount of time; to additional portions (10 g each) were added an inhibitor (0.25 mmol; Sigma-Aldrich, Milwaukee, Wis. or TCI), and these were also heated to 50° C. for the same amount of time. All were then cooled to room temperature prior to analysis. Inhibitors were purchased from TCI or Aldrich.

Mixtures of DCOIT and anisole in binders, either freshly prepared or aged several days at elevated temperature in a closed vial, were diluted at the ratio of 0.5 mL sample to 5 mL acetonitrile, to which were then added aqueous AlCl₃ (0.01 M, 100 mL) and HCl (12.1 M, 8 mL). After shaking to mix thoroughly and allowing to settle for a few hours, the samples were then filtered through a 0.45 micron filter and analyzed by HPLC photodiode array detector (210-500 nm) to measure anisole and DCOIT in each sample.

Percent degradation inhibited was calculated by comparing the DCOIT and anisole peak areas remaining in sample with a given additive to those without which had been heated and kept cool, ((DCOIT/Anisole)inhibitor,50° C.−(DCOIT/Anisole)no additive,50° C.) /((DCOIT/Anisole)no additive,RT−(DCOIT/Anisole)no additive,50° C.)*100%, such that an amount of DCOIT remaining equal to the sample held at room temperature would give an inhibition of 100%, and an amount remaining equal to the sample held at 50° C. would give an inhibition of 0%. Some additives accelerated the degradation, giving a negative value for inhibition, and some apparently worked better than holding at room temperature, giving values above 100%.

TABLE 1
Anhydrides in RHOPLEX ™ HG-706 resin
(0.25 mmol additive, 0.4 mmol DCOIT)

| Additive | % degradation inhibited |
|---|---|
| Phthalic anhydride | 112% |
| Maleic anhydride | 98% |
| Succinic anhydride | 117% |
| Boc anhydride | 36% |
| Poly(MVE-alt-MA) | 50% |
| 2,3-Dichloromaleic anhydride | 75% |
| Pyromellitic dianhydride | 95% |
| Decanoic anhydride | 33% |
| Hexadecanoic anhydride | 7% |
| Benzoic anhydride | 40% |
| Itaconic anhydride | 52% |
| 1,8-Naphthalic anhydride | 37% |
| 1,2-Cyclohexanedicarboxylic anhydride | 40% |
| 2,3-Pyridinedicarboxylic anhydride | 49% |
| 1,2,4-Benzenetricarboxylic anhydride | 61% |
| Poly(S-co-MA) | 56% |
| Diacetyl tartaric anhydride | 58% |
| Camphoric anhydride | 58% |
| Hexadecenylsuccinic anhydride | 68% |
| Trimellitic anhydride chloride | 73% |

From these data, phthalic anhydride, maleic anhydride, succinic anhydride, and pyromellitic dianhydride all performed extremely well.

TABLE 2
Phosphates, Sulfates, and Borates in RHOPLEX ™ in HG-706 resin
(0.25 mmol additive, 0.4 mmol DCOIT)

| Additive | % degradation inhibited |
|---|---|
| Phenylphosphonic acid | 45% |
| Sodium dodecyl sulfate | 8% |
| Triphenyl phosphate | 35% |
| Bromophenol blue | 60% |
| Sodium octadecyl sulfate | 28% |
| Sodium octyl sulfate | 32% |
| Sodium methyl sulfate | −16% |
| Sodium tridecyl sulfate | 4% |
| Triphenyl borate | −26% |
| Boric anhydride | 27% |
| Trihexadecyl borate | −99% |
| Pentafluorophenyl acetate | 49% |

Bromophenol blue and phenylphosphinic acid were the best performing stabilizers in this group.

TABLE 3
Other Organic Electrophiles in RHOPLEX ™ HG-706 resin
(0.25 mmol additive, 0.4 mmol DCOIT)

| Additive | % degradation inhibited |
|---|---|
| 2-Bromo-2-nitro-1,3-propanediol | −225% |
| Phthalimide | 42% |
| Sodium chloroacetate | −49% |
| Meldrum's acid | 14% |
| 3,3′-Methylenebis(4-hydroxycoumarin) | 26% |
| Phthalic acid | 28% |
| TEMPO | −148% |
| Fmoc-Ser(tBu)-Dhbt | 71% |

The top candidates in the other electrophile category were phthalimide and Fmoc-Ser(tBu)-Dhbt

TABLE 3
Other Inorganic Electrophiles in RHOPLEX ™ HG-706 resin
Ratio of DCOIT/Anisole signal
(0.25 mmol additive, 0.4 mmol DCOIT)

| Additive | % degradation inhibited* |
|---|---|
| Sodium chloride | −99% |
| Cesium chloride | −14% |

The inorganic electrophiles did not prove to be fruitful in DCOIT stabilization in resin.

### Stability Evaluations in Paints:

Paints tested: Valspar Duramax Semi-Gloss Latex Exterior Paint (low VOC, 100% acrylic, purchased at Lowes store in Oaks, Pa.) and Sherwin-Williams Showcase White Satin Latex Interior Paint and Primer (100% acrylic, <50 g/L VOC; purchased at Lowe's store in Oaks, Pa.).

### 8 Week Stability Testing Method:

### Preparation of Solid Additives

Solid additives (Sigma-Aldrich, St. Louis, Mo.) were typically used neat or prepared via crushing, where a pestle was used to finely grind approximately 5 g of solid additive in a stone mortar. If additional steps were taken to reduce particle size, they are noted in the examples. Particle size (neat, crushed, or otherwise) was measured by suspending roughly 0.5 g additive in 10 mL of MilliQ water. The additive suspension was vigorously mixed by pipetting up and down before being added dropwise to a Beckman Coulter LS 13 320 Laser Diffraction Particle Size Analyzer. Once a high enough signal count was recorded, the method was allowed to run. The volume statistics were calculated over the range of 0.375-2,000

### Preparation of Samples for 8-Week Stability Test

The appropriate amount of solid additive was added to the paint. The container was closed and sealed before it was mixed with a Red Devil (model 5400) paint shaker for 5 minutes. BIOBAN™ 200 (20% DCOIT) was used as the source of DCOIT, and resin and paint samples were dosed with 0.1-0.12 wt % DCOIT. Biocide was added dropwise into the paint over the course of 2 min, upon which samples were capped and vortexed briefly. Resin samples were mixed according to the protocol above. Using a plastic pipette, 10 g of paint sample was transferred into a 10 mL head space vial (1 vial needed/time point tested; Fisher Scientific). A hand crimper was used to cap the vials before tape was added to ensure a full seal (Agilent Technologies). The vials were incubated at the appropriate temperature (in oven at 50° C. or at room temperature, protected from light). Samples were removed from their incubation conditions at designated time points and allowed to equilibrate to room temperature.

### Analysis of DCOIT Concentration

From the paint, 0.2 g of the sample was removed and diluted 1:20 into MeOH, sonicated, and passed through a 0.22 μm filter for analysis by high performance liquid chromatography (HPLC) with UV detection at 280 nm to determine DCOIT concentrations. % DCOIT remaining=([DCOIT]_{time×weeks}/[DCOIT]_{time 0})*100

TABLE 4
Phthalic anhydride as a stabilizer in paint
% DCOIT Remaining (0.11 wt % DCOIT to start in
Valspar semi-gloss exterior Paint at 50° C.)

| | Weight % in paint (particle size) | | |
|---|---|---|---|
| | | 0.2 wt % | 0.2 wt % |
| Time | 0 wt % | (872 μm) | (133 μm) |
| 0 | 105% | 100% | 100% |
| 1 | NA | 75% | 60% |
| 2 | 5% | 69% | 60% |
| 4 | 0% | 30% | 41% |
| 8 | 0% | 14% | 26% |

TABLE 5
Triphenylphosphate (TPP) as a stabilizer in paint
% DCOIT Remaining (0.1 wt % DCOIT to start in
Valspar semi-gloss exterior Paint at 50° C.)

| | wt % TPP in paint (Particle Size (um)) | | |
|---|---|---|---|
| | | | 3.4 wt % |
| | | | (33.2 wt % |
| Time | | 1.13% | dispersion) |
| (weeks) | 0 wt % | (285 μm) | (80 μm) |
| 0 | 105% | 100% | 100% |
| 2 | 5% | 62% | 63% |
| 4 | 0% | 51% | 41% |
| 6 | 0% | 39% | 27% |
| 8 | 0% | 29% | 17% |

TPP was prepared at a particle size of 285 μm by hand crushing. In order to obtain a particle size of 80 μm, a dispersion of TPP was prepared. To prepare the TPP dispersion, TPP and 1 wt % TERGITOL™ 15-S-40 in water were first heated separately to 60° C. To a 1 oz vial, 4 g of TERGITOL™ 15-S-40 in water was added, followed by 1 g of the heated TPP. The sample was mixed by vortexing for 1 min (setting 10). If agglomeration was observed, the sample was reheated to 60° C. in a heated water bath while it was mixed with a stir bar. The sample was then removed from the water bath and stirred (600 rpm) at room temperature. Coulter counting was used to measure the size.

TABLE 6
Phthalimide as a stabilizer in paint
% DCOIT Remaining (0.1 wt % DCOIT to start
in Valspar semi-gloss exterior Paint at 50° C.)

wt % phthalimide
(Particle Size (μm))

| Time | | 0.51 wt % |
|---|---|---|
| (weeks) | 0 wt % | (352.4 μm) |
| 0 | 105% | 100% |
| 2 | 5% | 70% |
| 4 | 0% | 57% |
| 6 | 0% | 63% |
| 8 | 0% | 63% |

TABLE 7
Phthalic anhydride as a stabilizer in Valspar
semi-gloss exterior Paint
% DCOIT Remaining (0.12 wt % DCOIT to start in
Valspar semi-gloss exterior Paint at 50° C.)

wt % phthalic anhydride
(Particle Size (μm))

| Time | | 0.205 wt % | 0.205 wt % |
|---|---|---|---|
| (Weeks) | 0 wt % | (872.3 μm) | (as supplied) |
| 0 | 105% | 100% | 100% |
| 2 | 5% | 60% | 69% |
| 4 | 0% | 60% | 30% |
| 6 | 0% | 41% | 20% |
| 8 | 0% | 26% | 14% |

TABLE 8
Phthalic anhydride as a stabilizer in Satin deep acrylic Paint
% DCOIT Remaining (0.12 wt % DCOIT to start in Sherwin-Williams
Showcase White Satin Latex Interior Paint and Primer at 50° C.)

wt % phthalic anhydride
(Particle Size (μm))

| Time | | 0.114 wt % |
|---|---|---|
| (Weeks) | 0 wt % | (as supplied) |
| 0 | 96% | 100% |
| 2 | 21% | 75% |
| 4 | 16% | 50% |
| 6 | 0% | 53% |
| 8 | 0% | 30% |

1. An aqueous composition comprising: (a) 4,5-dichloro-2-n-octyl-4-isothiazolin-3-one; and (b) an aromatic acid anhydride comprising from 6 to 30 carbon atoms.

2. The aqueous composition of claim 1 in which a mole ratio of the aromatic acid anhydride to 4,5-dichloro-2-n-octyl-4-isothiazolin-3-one is from 0.3:1 to 20:1.

3. The aqueous composition of claim 2 in which the aromatic acid anhydride is an anhydride of an aromatic carboxylic or dicarboxylic acid.

4. The aqueous composition of claim 3 in which the aromatic acid anhydride comprises from 7 to 15 carbon atoms.

5. The aqueous composition of claim 4 in which the aqueous composition further comprises a dispersed acrylic or acrylic-styrenic polymer.

6. An aqueous composition comprising: (a) 4,5-dichloro-2-n-octyl-4-isothiazolin-3-one; and (b) a solid aliphatic acid anhydride comprising from 4 to 32 carbon atoms.

7. The aqueous composition of claim 6 in which a mole ratio of the solid aliphatic acid anhydride to 4,5-dichloro-2-n-octyl-4-isothiazolin-3-one is from 0.3:1 to 20:1.

8. The aqueous composition of claim 7 in which the solid aliphatic acid anhydride is an anhydride of an aromatic carboxylic or dicarboxylic acid.

9. The aqueous composition of claim 8 in which the solid aliphatic acid anhydride comprises from 4 to 22 carbon atoms.

10. An aqueous composition comprising: (a) 4,5-dichloro-2-n-octyl-4-isothiazolin-3-one; and (b) an aromatic imide comprising from 6 to 30 carbon atoms.
